(19) United States
(12) Patent Application Publication
(10) Pub. No.: US 2026/0216044 A1
(43) Pub. Date: Jul. 30, 2026

(54) COMPOSITION WITH CHITOSAN

(71) Applicant: L'OREAL, Paris (FR)

(72) Inventors: Christophe KUSINA, Chevilly Larue (FR); Nathalie BOILEAU, Chevilly Larue (FR); Danielle LE VERGE, Chevilly Larue (FR); Géraldine LEREBOUR, Chevilly Larue (FR); Angelina ROUDOT, Chevilly Larue (FR); Isabelle TERRISSE, Chevilly Larue (FR)

(73) Assignee: L'OREAL, Paris (FR)

(21) Appl. No.: 19/139,641

(22) PCT Filed: Dec. 19, 2023
(86) PCT No.: PCT/EP2023/086529

§ 371 Date: Jun. 16, 2025

(30) Foreign Application Priority Data
Dec. 19, 2022 (FR) 2213762

(51) Int. Cl. A61K 8/73; A61Q 1/02; A61Q 5/00; A61Q 19/00

(52) U.S. Cl. CPC A61K 8/736; A61Q 1/02; A61Q 5/00; A61Q 19/00; A61K 2800/43

(57) ABSTRACT

The present invention relates to a cosmetic composition comprising, in a physiologically acceptable aqueous medium:
(a) at least 0.01% by weight based on the total weight of the composition, native chitosan having a molecular weight strictly greater than 3000 daltons, this quantity being strictly less than 15% by weight,
(b) at least one alpha hydroxy acid;
(c) from 3 to 50% at least one pigmentary coloring material with respect to the total weight of the composition, and
(d) at least one polyol;
wherein the weight ratio between polyol(s) and chitosan is between 0.1 and 6.

The present invention relates to a cosmetic composition comprising, in a physiologically acceptable aqueous medium:
(a) at least 0.01% by weight based on the total weight of the composition, native chitosan having a molecular weight strictly greater than 3000 daltons, this quantity being strictly less than 15% by weight,
(b) at least one alpha hydroxy acid;
(c) from 3 to 50% at least one pigmentary coloring material with respect to the total weight of the composition; and
(d) at least one polyol;
wherein the weight ratio between polyol(s) and chitosan is between 0.1 and 6.

It also relates to a method for making up and/or treating the skin and/or keratin appendages, wherein the composition according to the invention is applied to the skin and/or keratin appendages.

There are numerous cosmetic compositions for which it is required that the deposited film, after application to keratin materials, should last well. Mention can be made for example of lipsticks or nail varnishes. In order to obtain such a result, it is possible to combine particular raw materials, in particular film-forming agents. However, the presence of such agents for the film to last well on keratin materials may lead to desiccating compositions.

Moreover, it is often sought to obtain compositions that cover well.

The formulator therefore seeks raw materials and/or systems making it possible to obtain fluid compositions (i.e. ones that flow) the deposit of which covers well and is homogeneous and strong (i.e. that lasts well).

In addition, there is a need for compositions the make-up properties of which, in particular lasting and covering well, are improved but moreover keeping comfort during application.

Moreover, formulating cosmetic products comprising high proportions of natural raw materials or raw materials of natural origin represents one of the new challenges for satisfying the expectations of consumers. It is therefore desirable for the compositions proposed to make it possible to keep a high proportion of natural raw materials or raw materials of natural origin.

An object of the present invention is therefore to provide a solution to the above problems.

The aim of the present invention is to propose aqueous cosmetic properties having improved make-up properties, in particular in terms of coverage and homogeneity, but also lasting well.

After application, these compositions leave a homogeneous film-forming deposit that covers well and has good resistance to wear. The films formed are adhesive and cohesive, and cover and last well. In particular, they have improved resistance to friction.

The present invention relates to a cosmetic composition, in particular for make-up and/or treatment of the skin and/or lips, comprising, in a physiologically acceptable aqueous medium:
(a) at least 0.01% by weight based on the total weight of the composition, native chitosan having a molecular weight strictly greater than 3000 daltons, this quantity being strictly less than 15% by weight,
(b) at least one alpha hydroxy acid;
(c) from 3 to 50% at least one pigmentary coloring material with respect to the total weight of the composition;
(d) at least one polyol, and wherein the weight ratio between polyol(s) and chitosan is between 0.1 and 6.

“Physiologically acceptable” refers to a medium compatible with keratin materials.

Another object thereof is a method for making up and/or caring for the skin and/or the keratin appendages, wherein the composition according to the invention is applied to the skin and/or the keratin appendages.

Preferably, the composition according to the invention is substantially free from (meth)acrylic polymer. Preferably, the composition according to the invention is substantially free from silicone.

By “substantially free from (meth)acrylic polymer” is meant that the composition comprises less than 1% by weight relative to the total weight of the composition, preferably less than 0.5% by weight, preferably less than 0.3% by weight, preferably 0.1% by weight of (meth)acrylic polymer. Preferably, the composition is completely free from (meth)acrylic polymer. By (meth)acrylic polymer is meant a polymer comprising at least one acrylic acid monomer or at least one methacrylic acid monomer.

By “substantially free from silicone” is meant that the composition comprises less than 1% by weight relative to the total weight of the composition, preferably less than 0.5% by weight, more preferably less than 0.3% by weight, preferably 0.1% by weight, of silicone. Preferably, the composition is completely free from silicone. By silicone is meant any silicone compound.

### Chitosan

The composition according to the invention comprises at least 0.01%, with respect to the total weight of the composition, at least one native chitosan having a molecular weight strictly greater than 3000 daltons (3 kDa, 1 Da=1 g/mol).

The quantity of native chitosan is also strictly less than 15% by weight with respect to the total weight of the composition.

Preferably, the native chitosan has a molecular weight greater than or equal to 10 kDa, preferably greater than or equal to 15 kDa, preferably greater than or equal to 20 kDa. Preferably, the native chitosan has a molecular weight of between 10 kDa and 2 MDa preferably between 15 kDa and 1.5 MDa, preferably between 20 kDa and 300 kDa, preferably between 20 kDa and 150 kDa.

Chitosan (or chitosane) is very widespread in nature. It is signaled only in the exoskeletons of certain insects such as termite queens and in the cell walls of a particular class of mushroom, zygomycetes.

Chitosan is obtained by the deacetylation of chitin. Chitin is a polysaccharide composed of several N-acetyl-D-glucosamine units connected together by a bond of the β (1,4) type.

The ideal chemical structure of chitosan is a chain of p-D-glucosamine monomers connected by a glycoside bond (1→4).

“Chitosan” according to the invention means any copolymer formed by N-acetyl-D-glucosamine and D-glucosamine constituent units, the degree of acetylation of which is less than 90%. Chitosan consists of glucosamine sugar units (deacetylated units) and N-acetyl-D-glucosamine units (acetylated units) connected together by bonds of the β (1,4) type and constitutes a polymer of the Poly(N-acetyl-D-glucosamine)-poly(D-glucosamine) type.

Preferably, the degree of acetylation of chitosan is less than or equal to 50%, preferably less than or equal to 35%, preferably less than or equal to 25%, preferably less than or equal to 15%.

The degree of acetylation is the percentage of acetylated units with respect to the total number of units, it can be determined by Fourier transform infrared (FTIR) spectroscopy or by titration by a strong base.

The chitosan of the invention is preferably a polysaccharide prepared from a fungal origin. It is in particular extracted and purified from safe and abundant food or biotechnological fungal sources such as Agaricus bisporus or Aspergillus niger.

The chitosan of the invention preferably comes from the mycelium of a mushroom of the Ascomyceta type and in particular Aspergillus niger and/or of a Basidiomyceta mushroom, and in particular Lentinula edodes (shiitake) and/or Agaricus bisporus. Preferably the mushroom is Aspergillus niger.

The chitosan may be of GMO origin, but is preferably of non-GMO origin.

The chitosan according to the invention is native, i.e. it is not modified. In particular it does not contain any chemical modification.

One method for preparing chitosan is the one described in the application WO03068824.

The chitosan used in the invention is preferably in powder form. It is in particular marketed by Kitozyme under the name Kiosmetine or Kionutrime.

The chitosan is preferably present in a quantity ranging from 0.01% to 14% by weight, preferably from 0.1% to 14% by weight, preferably from 0.1% to 12% by weight, preferably from 0.2% to 7% by weight, and preferably from 0.25% to 5% by weight with respect to the total weight of the composition.

### Alpha Hydroxy Acid (AHA)

The composition according to the present invention also comprises (b) at least one alpha hydroxy acid (AHA).

Alpha hydroxy acid means, according to the present invention, a carboxylic acid having at least one hydroxy function occupying an alpha position on said acid (carbon adjacent to a carboxylic acid function). This acid may be in the final composition in the form of free acid and/or in the form of one of the associated salts thereof (salts with an organic base or an alkali in particular), in particular according to the final pH imposed on the composition.

The α-hydroxy acids (alpha hydroxy acids or AHA) are for example selected from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; mandelic acid; phenyllactic acid; gluconic acid; galacturonic acid; aleuritic acid; ribonic acid; tartronic acid; tartaric acid; malic acid; fumaric acid; salts and mixtures thereof.

According to a preferred embodiment, the alpha hydroxy acid (b) is selected from lactic acid, gluconic acid, citric acid, malic acid, tartaric acid and salts thereof. More particularly, the alpha hydroxy acid is selected from lactic acid, citric acid and salts and mixtures thereof.

The alpha hydroxy acid or acids (b) can be present in a quantity ranging for example from 0.001 to 10% by weight, from 0.005 to 5% by weight and preferably from 0.01% to 2.5% by weight, with respect to the total weight of the composition.

### Pigmentary Dyes

The composition according to the invention comprises from 3% to 50% at least one pigmentary dye (c) with respect to the total weight of the composition. This dye is thus selected from powder dyes such as mineral pigments, nacres and organic pigments.

The term “pigments” should be understood to mean white or colored, mineral or organic s particles, which are insoluble in an aqueous solution and are intended for coloring the composition and/or the resulting deposit.

A pigmentary dye is a compound that has a color index (CI).

The term “pigments” should be understood to mean white or colored, mineral or organic particles, insoluble in an aqueous medium and intended for coloring the composition and/or the resulting deposit.

Preferably, the pigmentary dye of the invention has a refractive index of at least 1.9 at 25° C.

The refractive index of the pigmentary dye of the invention is preferably comprised between 1.9 and 4. Especially, the pigmentary dye is different from fillers; preferably the pigmentary dye is different from talc.

The dyes may be present, in the composition, at a content ranging from 3% to 45% by weight, with respect to the weight of the composition, preferably from 4% to 30% by weight, preferably from 5% to 20% by weight, preferably from 6% to 15% by weight.

### Mineral Pigments

According to one particular embodiment, the pigments (c) used according to the invention are selected from mineral pigments.

“Mineral pigment” means any pigment that complies with the definition in Ullmann's Encyclopedia in the Inorganic Pigment chapter. Mention can be made, among the mineral pigments useful in the present invention, of zirconium or cerium oxides, along with zinc, iron (black, yellow or red) or chromium oxides, manganese violet, ultramarine blue, chromium hydrate and iron blue, titanium dioxide and metallic powders such as aluminum powder and copper powder. The following mineral pigments can also be used: Ta2O5, Ti3O5, Ti2O3, TiO, ZrO2 in a mixture with TiO2, ZrO2, Nb2O5, CeO2, ZnS.

The size of the pigment useful in the context of the present invention is in general greater than 100 nm and may range up to 10 μm, preferably from 200 nm to 5 μm, and more preferentially from 300 nm to 1 μm. According to a particular embodiment of the invention, the pigments have a size characterized by a D50 greater than 100 nm and may range up to 10 μm, preferably from 200 nm to 5 μm, and more preferably from 300 nm to 1 μm. The sizes are measured by measured by static diffusion of light by means of a commercial MasterSizer 3000@granulometer from Malvern, making it possible to apprehend the granulometric distribution of all the particles over a wide range that can range from 0.01 μm to 1000 μm. The data are processed on the basis of the classic Mie scattering s theory. This theory is the most adapted for size distributions ranging from submicron to multi-micron, and makes it possible to determine an “effective” diameter of particles. This theory is in particular described in the work by Van de Hulst, H. C., “Light Scattering by Small Particles”, Chapters 9 and 10, Wiley, New York, 1957. D50 represents a maximum size that 50% by volume of the particles have.

In the context of the present invention, the mineral pigments are more particularly iron oxide and/or titanium dioxide.

As mineral pigments that can be used in the invention, nacres can also be cited.

The term “nacres” should be understood to mean iridescent or non-iridescent colored particles of any shape, which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface whereof are superposed at least two successive layers of metal oxides and/or of organic dyes. By way of example of nacres, mention may also be made of natural mica coated with titanium oxide, with iron oxide, with natural pigment or with bismuth oxychloride. The nacres may more particularly possess a yellow, pink, red, bronze, orange, brown and/or copper color or glint.

Among the pigments that can be used according to the invention, mention can also be made of those with an optical effect different from a simple conventional hue effect, i.e. a unified and stabilized effect of the kind produced by conventional dyes, such as, for example, monochromatic pigments.

For the purpose of the invention, “stabilized” means absence of an effect of variability of color with the angle of observation or in response to a temperature change. For example, this material may be selected from particles having a metallic glint, goniochromatic coloring agents, diffracting pigments, thermochromatic agents, optical brighteners, and also fibers, in particular of the interference type. Of course, these various materials may be combined so as to provide the simultaneous manifestation of two effects, or even a new effect in accordance with the invention.

According to one particular embodiment, the composition according to the invention comprises at least one non-coated pigment.

### Organic Pigments

According to another embodiment of the invention, the pigmentary dye (c) is an organic, synthetic or natural pigment or one of natural origin. “Organic pigment” means any pigment that complies with the definition in Ullmann's Encyclopedia in the Organic Pigment chapter. The organic pigment can in particular be selected from the compounds nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, quinophthalone.

The organic pigment or pigments (c) can be selected for example from carmine, carbon black, aniline black, melanin, azo yellow, quinacridone, phthalocyanine blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indole or phenolic derivatives as described in patent FR 2 679 771.

The pigments can also be in the form of composite pigments as described in patent EP 1 184 426. These composite pigments may be composed in particular of particles including an inorganic core covered at least partially with an organic pigment and at least one binder for fixing the organic pigments to the core.

The pigment may also be a lacquer.

The term lacquer means non-solubilized dyes adsorbed on insoluble particles, the assembly thus obtained remaining insoluble during use.

The inorganic substrates onto which the dyes are adsorbed are for example alumina, silica, calcium sodium borosilicate or calcium aluminum borosilicate, and aluminum.

Among the organic dyes, mention can be made of cochineal carmine. The products known under the following names can also be cited: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090). By way of examples of lacquers, the product known by the name D&C Red 7 (CI 15 850:1) can be cited.

### Polyols

The composition according to the present invention also comprises (d) at least one polyol.

The term “polyol” designates here an alcohol having two hydroxy groups or more and does not include a saccharide or a derivative thereof. The derivative of a saccharide includes a sugar alcohol obtained by reducing one or more carbonyl groups of a saccharide, as well as a saccharide or a sugar alcohol wherein the hydrogen atom or atoms in one or more hydroxy groups thereof has (have) been replaced by at least one substituent such as an alkyl group, a hydroxyalkyl group, an alkoxy group, an acyl group or a carbonyl group.

The polyol (d) may be a C₂₋₂₄ polyol, preferably a C₂₋₉ polyol, comprising at least 2 hydroxy groups, and preferably 2 to 5 hydroxy groups.

The polyol (d) can be a natural or synthetic polyol. The polyol can have a linear, branched or cyclic molecular structure.

The polyol (d) can be selected from glycerins and derivatives thereof, as well as glycols and derivatives thereof. The polyol can be selected from the group consisting of glycerin, diglycerin, polyglycerin, ethylene glycol, diethylene glycol, propylene glycol, dipropylene glycol, butylene glycol, pentylene glycol, hexylene glycol, 1,3-propanediol, 1,4-butanediol, 1,5-pentanediol, and polyethylene glycols, particularly having from 5 to 50 ethylene oxide groups and mixtures thereof; more particularly selected from glycerin, propylene glycol, dipropylene glycol, butylene glycol, hexylene glycol and mixtures thereof.

It is preferable for the polyol (d) to be glycerin.

The polyol (d) may be present in the composition according to the invention in a content ranging from 0.01% to 50% by weight, in relation to the total weight of the composition, preferably ranging from 0.05% to 30% by weight, preferably ranging from 0.08% to 20% by weight, preferably ranging from 0.1 to 15% by weight, preferably from 0.1% to 12% by weight, preferably from 0.1% to 10% by weight, preferably from 0.2% to 8% by weight, preferably from 0.2% to 5% by weight, preferably from 0.2% to 3% and preferentially ranging from 0.2% to 2% by weight.

Preferentially, when the proportion of chitosan in the composition is greater than or equal to 2% by weight with respect to the total weight of the composition, the polyol (d) is present in a proportion ranging from 0.5% to 15% by weight, preferably from 1% to 12% by weight, preferably from 3% to 10.

According to the invention, the weight ratio between the polyol(s) and the chitosan (i.e. polyol(s) to chitosan weight ratio) is between 0.1 and 6. Preferably, it is between 0.1 and 5, preferably between 0.2 and 5, preferably between 0.2 and 4.5, preferably between 0.5 and 2.

### Physiologically Acceptable Aqueous Medium

The composition according to the invention comprises a physiologically acceptable aqueous medium. Said medium comprises water.

The water used can be sterile demineralized water and/or floral water such as rose water, cornflower water, chamomile water or linden water, and/or a spring or natural mineral water.

The composition preferably comprises at least 5% by weight water with respect to the total weight of the composition, preferably at least 10% by weight, preferably at least 20% by weight, preferably at least 30% by weight, preferably at least 40% by weight.

The composition preferably comprises from 5% to 95% by weight water with respect to the total weight of the composition, more preferentially from 10% to 85%, even more preferentially from 20% to 80%, even more preferentially from 25% to 70%, even more preferentially from 28% to 60%, even more preferentially from 30% to 50%.

The aqueous phase can also comprise at least one non-ionic surfactant.

Preferably, in this case, the non-ionic surfactant can be selected from the non-ionic surfactants known from the prior art, in particular fatty acid and polyglycerol esters, sugar esters, poloxamers, polysorbates and mixtures thereof.

The non-ionic surfactant is preferably selected from fatty acid and polyglycerol esters and sugar esters.

### PH of the Composition

The composition according to the invention has a pH less than or equal to 7, preferably less than or equal to 6.5, preferably less than or equal to 6.3. Advantageously, s the pH of the composition is between 3 and 6.3, preferably between 4 and 6.3.

Preferably, the cosmetic composition according to the invention comprises at least one base.

The base is particularly used to adjust the final pH of the composition to between 3 and 6.3.

The base can be chosen from mineral bases such as for example alkali metal hydroxides, sodium hydroxide, potassium hydroxide.

Preferably, the base of the composition is an alkali metal hydroxide, preferably sodium hydroxide or potassium hydroxide. This is because these bases are more advantageous than nitrogenous bases, such as triethanolamine, on color stabilization (i.e. less yellowing).

The composition according to the invention can comprise at least one base at an active substance content ranging from 0.5% to 10% by weight, with respect to the total weight of the composition, particularly from 1% to 5% by weight, preferably ranging from 1% to 4% by weight.

The composition according to the invention can be obtained conventionally by those skilled in the art.

The following examples will make it possible to understand the invention better, without being in any way limitative. The raw materials are referred to by the chemical or INCI name thereof. The amounts indicated are as a % by weight of raw materials with respect to the total weight of the composition (% w/w), unless specified otherwise.

### EXAMPLES

### Preparation of the Compositions According to the Invention and Comparative Compositions

In the following examples, each formula was prepared in accordance with the protocol described below:
Under mechanical stirring with minimal vortex and at ambient T°,
   Disperse the chitosan and water under mechanical stirring with minimal vortex.
   Add the appropriate quantity of acid to solubilize the chitosan and form a more viscous solution.
   Add the pigments, then the polyols, then the surfactants (if present?).
   The mixture can then be passed through a triple-roll mill if necessary.

The chitosan is judged to be solubilized when the solution loses transparency. For this purpose the transmittance of the composition is measured by means of the Turbiscan LAB and the Turbisoft LAB software.

### Example 1: Evaluations of the Effects of the Various Ingredients

The formulas F1 to F11 are prepared according to the above protocol.

Protocol Testing the Film-Forming and Peelability Aspect of the Formulas after Deposition

In an aluminum capsule 9.5 cm in diameter, deposit 10 g of solution and spread homogeneously over the entire surface. The deposit is left to dry for 24 hrs at 32° C. on a hotplate. The sample is next evaluated to the touch initially. If the sample is liquid and remains stuck to the finger, it is considered that the sample is not capable of forming a film. In the case where the sample can form a film, a second evaluation consisting of detaching the sample from the aluminum capsule by means of a spatula. If the experimenter manages this, it will be said that the deposit is peelable.

The results are described below in table 1.

The appearance is said to be compliant if the appearance of the preparation is fluid and can flow. The appearance will be non-compliant if the formulation is solid or in the form of a gum or is unable to flow.

The sensorial property is defined by spreading with a finger over a square of the skin of the arm 2.54 cm square. If the spreading is not fluid and clearly gives rise to sensations of irregularities of the bumpy texture or fragile solid type, it will be said that the sensorial property is non-compliant. If the spreading is fluid it will be said that the sensorial property is compliant.

In the table OK=compliant, NO=non-compliant, F5 and F6=Intermediate.

Homogeneity is described on the visual aspect of the preceding film. If it includes visible non-homogeneities of thickness, it will be said that the film is non-homogenous. If the film is visually of constant thickness, it will be said that the film is homogenous.

TABLE 1

| | | | | Appearance | | | | |
|---|---|---|---|---|---|---|---|---|
| | | | | of the | | | | Homogeneity |
| | Chitosan | AL | Gly | composition | Film- | | Sensorial | of the film |
| Formula | (% w/w) | (% w/w) | (% w/w) | (bulk) | forming | Peelable | properties | on the skin |
| F1 | 0.1 | 0.05 | | COMPLIANT | OK | NONE | OK | homogeneous |
| F2 | 0.5 | 0.25 | | COMPLIANT | OK | OK | OK | homogeneous |
| F3 | 10 | 5 | | COMPLIANT | OK | OK | OK | homogeneous |
| F4 | 15 | 7.5 | | NON- | OK | OK | NO since | Non- |
| | | | | COMPLIANT | | | solid | homogeneous |
| | | | | Elastic solid | | | | |
| F5 | 14 | 7 | | COMPLIANT | OK | OK | Exfoliates | Non- |
| | | | | | | | with | homogeneous |
| | | | | | | | difficulty | |
| | | | | | | | but OK | |
| F6 | 13 | 6.5 | | COMPLIANT | OK | OK | Exfoliates | Non- |
| | | | | | | | with | homogeneous |
| | | | | | | | difficulty | |
| | | | | | | | but OK | |
| F7 | 12 | 6 | | COMPLIANT | OK | OK | OK | homogeneous |
| F8 | 5 | 2.5 | 20 | COMPLIANT | OK | OK | OK | Homogeneous |
| F9 | 5 | 2.5 | 30 | COMPLIANT | OK | NONE | OK | Non- |
| | | | | | | | | homogeneous |
| F10 | 5 | 2.5 | 40 | COMPLIANT | NONE | NONE | OK | Non- |
| | | | | | | | | homogeneous |
| F11 | 5 | 2.5 | 50 | COMPLIANT | NONE | NONE | NO since | Non- |
| | | | | | | | solid | homogeneous |

In the following table and hereinafter
AL = lactic acid
Gly = Glycerin

Formulas F1 and F2: The minimum dose of chitosan to form a film detectable by this test is 0.1%. The quantity of 0.5% makes it possible to obtain a peelable film.

Formula F4: Chitosan must in all cases be present in a quantity strictly less than 15% by weight, since the formula is then no longer applicable and the appearance of the composition (bulk) is no longer fluid.

Formulas F8 to F10: the weight ratio between polyol and chitosan is ideally less than or equal to 6 (F9), since beyond that the deposits are no longer homogeneous (F10 and F11), preferentially equal to 4 to obtain a very homogeneous film (F8). Non-homogeneity stems from the inability to work/mix/apply the material formed.

### Example 2: Protocol for Evaluating Modifications to the Mechanical Properties of the Films to Improve Comfort

The formulas C1 to C6, A1 and A2 are prepared.

In an aluminum capsule 9.5 cm in diameter, deposit 10 g of solution and spread homogeneously over the entire surface. The deposit is left to dry for 24 hrs at 32° C. on a hotplate.
Cut 3 test pieces 20 mm long x 6 mm wide using a cutting die.
Measure the thickness of the test pieces by means of a Palmer micrometer.
Place the test piece in the jaws of a TA.XT Plus texturometer from Stable Micro Systems and, using the Exponent software, initiate measurement in order to obtain the strength (g) and length at break (mm).
TA Setting: speed 0.1 or 0.3 mm/s

$$\% deformation = elongation / initial size * 100 = ( elongation at break - initial size ) / initial size * 100 stress = strength ( N ) / surface ( m ) = strength ( g ) * g / width * thickness$$

TABLE 2

| | Chitosan | | | % Mean | | | |
|---|---|---|---|---|---|---|---|
| | (100 kDa) | AL | Glycerin | elongation | Error on | Mean stress | Error on |
| Formula | (% w/w) | (% w/w) | (% w/w) | at break | elongation % | at break | stress |
| C1 | 5 | 2.5 | 0 | 4.8 | 0.9 | 46 | 7.5 |
| C2 | 5 | 2.5 | 0.1 | 4.3 | 1.5 | 34.8 | 11.4 |
| C3 | 5 | 2.5 | 0.5 | 8.0 | 0.4 | 18.5 | 10.7 |
| C4 | 5 | 2.5 | 1 | 50.0 | 25.6 | 16.4 | 4.6 |
| C5 | 5 | 2.5 | 5 | 593.2 | 59.8 | 1.5 | 0.2 |
| C6 | 5 | 2.5 | 20 | 723.3 | 85.6 | 0.1 | 0.0 |

These results make it possible to determine the minimum dose of glycols with respect to the chitosan with regard to the technical problem of comfort. Having a softer film, and therefore one that has longer elongation at break, improves comfort on deposition.

Without glycerin, the chitosan film remains rigid and uncomfortable on the skin (C1). The change in rigidity of the film is evaluated with the above measurements and shows that, as from a weight ratio of polyol to chitosan equal to 1:10 (C3) the suppling effects are perceptible. This suppling is essential for comfort.

Formula A1 is comparative (marked by a star), while formula A2 is according to the invention.

TABLE 3

| | Chitosan | | | Red | % Mean | | | |
|---|---|---|---|---|---|---|---|---|
| | (100 kDa) | AL | Glycerin | pigment | elongation | Error on | Mean stress | Error on |
| Formula | (% w/w) | (% w/w) | (% w/w) | (% w/w) | at break | elongation % | at break | stress |
| A1* | 5 | 2.5 | | 15 | 1.3 | 0.1 | 18.2 | 1.2 |
| A2 | 5 | 2.5 | 10 | 15 | 208.5 | 7.7 | 1.0 | 0.1 |

Example A2 shows the impact of the polyol on the flexibility of the film in the presence of pigment.

Formulas B, C and E according to the invention are prepared.

Mechanical properties are measured as indicated above.

TABLE 4

| | Chitosan | | | | | | Red |
|---|---|---|---|---|---|---|---|
| | (100 kDa) | AL | AC | PG4 | HexG | ButG | pigment |
| Formula | (%. w/w) | (%. w/w) | (%. w/w) | (%. w/w) | (%. w/w) | (%. w/w) | (%. w/w) |
| B | 5 | 2.5 | | 10 | | | 15 |
| C | 5 | 2.5 | | | 10 | | 15 |
| E | 5 | | 6 | | | 10 | 15 |

PG4: Polyglycerin-4 sold under the name (PURE VEGETABLE POLYGLYCERINE-4 by SPIGA
AC: Citric acid
HexG: Hexylene glycol sold by ARKEMA
ButG: Butylene Glycol sold by DAICEL

TABLE 5

| | % Mean | | % Mean | |
|---|---|---|---|---|
| | elongation | Error on | stress at | Error on |
| Formula | at break | elongation % | break | stress |
| B | 135.9 | 8.6 | 6.3 | 0.7 |
| C | 6 | 0.9 | 18.3 | 1.8 |
| E | 11.9 | 1.3 | 5.7 | 0.4 |

Examples B, C and E show the impact of the polyols on the flexibility of the film in the presence of pigment. Example K further shows that citric acid (AHA) can be used instead of lactic acid.

### Example 3: Protocol for Evaluating Resistance to Rubbing when Dry

Some compositions are evaluated for their resistance to rubbing, by colorimetric measurements on dry film before and after abrasion, a test the protocol of which is detailed below.

### 3.1. Protocol for Spreading Compositions in a Film

The products are spread on a spreading bench (Elcometer 4340 Applicator) making it possible to adjust its speed as well as the distance over which it takes place. The bench is equipped with an aspiration system connected to a pump so that the support where the spreading is done does not move. Contrast charts with non-varnished black background and white background are used (1 byko-chart, uncoated N2A, code 2831). As for the spreading thickness, this is adjustable by means of the square spreader deposited on the support so as to spread by levelling when the platform is started up. Each edge of the spreader makes it possible to spread with a different thickness ranging from 25 μm to 200 μm. The thickness selected is 50 μm in order to approach a thickness of the film in vivo. A weight of 960 g is added on top of the spreader during spreading. The spreading speed is adjusted at 1 in/sec, i.e. 2.54 cm/s. The films are dried for 24 hrs at ambient temperature and humidity (50% RH).

TABLE 6

| | | | | Native | |
|---|---|---|---|---|---|
| | Chitosan | AL | Glycerin | pigments | |
| Formula | (100 kDa) | (% w/w) | (% w/w) | (% w/w) | Wetting |
| Comparative | 0.1 | 0.08 | 0 | 15 | No, |
| composition | | | | | therefore |
| | | | | | non- |
| | | | | | homo- |
| | | | | | geneity |
| Composition | 0.1 | 0.08 | 1 | 15 | Yes |
| according to | | | | | |
| the invention | | | | | |

The results are shown in table 3. Some compositions cannot be spread to perform the contrast ratio (CR) tests that follow, it is said that they dewet. This dewetting causes non-homogeneities of deposition. These formulas therefore do not meet the criteria for solving the technical problem of homogeneity and coverage because of dewetting thereof.

On the other hand, the composition according to the invention has good wettability.

### 3.2. Protocol for Measuring the Contrast Ratio to Objectify the Coverage

The color is measured by Konica Minolta CM-700d spectrophotometer. Measurement with contact guarantees the absence of light pollution.

Settings selected: Aperture 8 mm; Uncertainty: 0.04; SCI/SCE measurement; Geometry d/8°.

The color measurements on the two backgrounds (black background BB and white background WB) make it possible to characterize the coverage of a make-up foundation by calculating the contrast ratio (CR %) i.e. YBB/YWB×100, where YBB and YWB, where YBB and YWB are respectively the luminance values measured on black background and white background, this being higher, the better the coverage of the make-up foundation.

### Quantity of Pigments

Formula F12 is according to the invention, whereas formulas F13 to F16 are for comparison.

TABLE 7

| | | | | Native | | |
|---|---|---|---|---|---|---|
| | Chitosan | AL | Glycerin | pigments | TA | |
| Formula | (100 kDa) | (% w/w) | (% w/w) | (% w/w) | (PG10L) | CR |
| F12 | 1 | 0.5 | 1 | 3 | 0.1 | 52 ± 2 |
| (invention) | | | | | | 50 μm |
| F13 | 1 | 0.5 | 1 | 2.5 | 0.1 | 44 ± 1 |
| | | | | | | 50 μm |
| F14 | 1 | 0.5 | 1 | 2 | 0.1 | 43 ± 1 |
| | | | | | | 50 μm |
| F15 | 1 | 0.5 | 1 | 1.5 | 0.1 | 35 ± 2 |
| | | | | | | 50 μm |
| F16 | 1 | 0.5 | 1 | 1 | 0.1 | 19 ± 1 |
| | | | | | | 50 μm |

PG10L = surfactant (PG10 laurate)

In order to achieve a minimum coverage fixed at 50%, it is necessary to have a minimum of 3% pigments.

### Nature of the Chitosan

Formulas F19 to F20 are according to the invention, whereas formulas F17 to F18 are for comparison.

TABLE 8

| | | Molecular | | | Native | | |
|---|---|---|---|---|---|---|---|
| | Chitosan | weight | AL | Glycerin | pigments | | |
| Formula | (% w/w) | (Da) | (% w/w) | (% w/w) | (% w/w) | AT | CR0 |
| F17 | 0 | | | 5 | 15 | 1 | 84 ± 4 |
| F18 | 5% | <3 kDa | | 5 | 15 | 1 | 89 ± 1 |
| F19 | 5% | 20 kDa | 2.5 | 5 | 15 | 1 | 95 ± 1 |
| (invention) | | | | | | | |
| F20 | 5% | 100 kDa | 2.5 | 5 | 15 | 1 | 103 ± 3 |
| (invention) | | | | | | | |

AT = surfactant (PG10 laurate)

Formulas F19 to F20 are the only ones that are significantly different from F17 (reference without chitosan) with regard to coverage in the deposit.

### 3.3. Protocol for Testing Resistance to Rubbing

The test for resistance to rubbing is implemented by colorimetric measurements on dry film before and after abrasion. Abrasion is implemented by attaching a fabric handkerchief strip (Chicopee® Veraclean™ Polish Plus) on the spreader edge at 25 μm. A weight of 960 g is added on top of the spreader during abrasion. The bench speed is set at 2.54 cm/s.

The color measurement before and after abrasion is done with the same spectrophotometric method as explained before.

In order to evaluate resistance to rubbing, the contrast ratio respectively before rubbing (CR Dry Deposit, %) and after abrasion (CR Rub Deposit Dry, %) is measured. The loss [(CR Rub Deposit Dry−CR Deposit Dry]/CR Deposit Dry]*100, as a percentage, quantifies the loss of coverage and indicates the resistance of the film to rubbing: the smaller this loss, the more the film is resistant to rubbing.

Each CR value therefore represents a mean of 6 measurements.

### Selection of Type of Chitosan

TABLE 9

| Formula | % Loss CR |
|---|---|
| F17 | 23.8 ± 2 |
| F18 | 26.3 ± 1 |
| F19 | 0 ± 1 |
| F20 | −0.3 ± 1 |

The formulas F19 to F20 are the only ones not to lose coverage.

### Quantity of Chitosan

Formula F27 is according to the invention, whereas formulas F22 to F26 are for comparison.

TABLE 10

| | Chitosan | | | | | |
|---|---|---|---|---|---|---|
| | (100 kDa) | AL | Glycerin | Native | | % Loss |
| Formula | (% w/w) | (% w/w) | (% w/w) | pigments | AT | CR |
| F17 | 0 | | 5 | 15 | 1 | 23.8 ± 2 |
| F22 | 0 | | 0 | 15 | 0.1 | 21.9 ± 1 |
| F23 | 0.001 | 0.0008 | | 15 | 0.1 | 24.6 ± 2 |
| F24 | 0.005 | 0.004 | | 15 | 0.1 | 23.9 ± 1 |
| F25 | 0.01 | 0.008 | | 15 | 0.1 | 5.6 ± 1 |
| F26 | 5 | 4 | | 15 | 0.1 | 0.8 ± 1 |
| F27 | 5 | 2.5 | 5 | 15 | 1 | −0.3± 1 |
| (invention) | | | | | | |

The comparative formulas F17 and F22 show that the presence of glycerin does not have any impact on coverage.

Comparing the formulas F23-F26 (comparative) and F27 (invention) shows that chitosan has an impact on coverage.

### 3.4. Test for Resistance to Oily Friction

The test consists in spreading 15 μL of formula on a disk 4 cm in diameter on the forearm and leaving the deposit to dry for 20 minutes. In parallel, 100 μL of oil (5022 caprylic/capric triglyceride) is deposited on a fabric handkerchief strip (Chicopee® Veraclean™ Polish Plus). Next the handkerchief is rubbed with an axial force of 200-300 grams and the stain on the handkerchief is evaluated.

TABLE 11

| | Chitosan | | Native | | | Oil |
|---|---|---|---|---|---|---|
| | (100 kDa) | AL | pigments | AT | Dipropylene | strength |
| Test | (% w/w) | (% w/w) | (% w/w) | PG10L | glycol | score |
| Comparative | 0 | | 15 | 1 | 12 | 1/5 |
| composition | | | | | | |
| Composition | 3 | 1.5 | 15 | 1 | 12 | 3/5 |
| according | | | | | | |
| to the | | | | | | |
| invention | | | | | | |

The positive bound having a score of 5/5 corresponds to a non-stained tissue while, if the tissue is completely colored, a score of 1/5 will be attributed.

Under these conditions the composition according to the invention performs so as to obtain a score of 3/5.

It can be seen that chitosan improves the resistance to oily friction of the deposits.

1. A cosmetic composition comprising, in a physiologically acceptable aqueous medium:
(a) at least 0.01% by weight based on the total weight of the composition, native chitosan having a molecular weight strictly greater than 3000 daltons, this quantity being strictly less than 15% by weight,
(b) at least one alpha hydroxy acid;
(c) from 3 to 50% at least one pigmentary coloring material with respect to the total weight of the composition;
(d) at least one polyol;
wherein the weight ratio between polyol(s) and chitosan is between 0.1 and 6.

2. The composition according to claim 1, which comprises from 0.01% to 14% by weight of the native chitosan with respect to the total weight of the composition.

3. The composition according to claim 1, wherein the native chitosan (a) has a molecular weight greater than or equal to 10 kDa, and/or the degree of acetylation of the chitosan is less than or equal to 50%.

4. The composition according to claim 1 wherein the chitosan (a) is extracted and purified from food or biotechnological fungal sources.

5. The composition according to claim 1 wherein the α-hydroxy acid (b) is selected from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; gluconic acid; mandelic acid; phenyllactic acid; gluconic acid; galacturonic acid; aleuritic acid; ribonic acid; tartronic acid; tartaric acid; malic acid; fumaric acid; salts and mixtures thereof.

6. The composition according to claim 1, wherein the alpha hydroxy acid (b) is present in a quantity ranging from 0.001 to 10% by weight with respect to the total weight of the composition.

7. The composition according to claim 1, wherein the dye (c) is selected from powder dyes such as mineral pigments, nacres and organic pigments.

8. The composition according to claim 1, wherein the polyol (d) is selected from glycerin, diglycerin, polyglycerin, ethylene glycol, diethylene glycol, propylene glycol, dipropylene glycol, butylene glycol, pentylene glycol, hexylene glycol, 1,3-propanediol, 1,4-butanediol, 1,5-pentanediol, the polyethylene glycols comprising 5 to 50 ethylene oxide groups and mixtures thereof.

9. The composition according to claim 1, wherein the polyol (d) is present in a content ranging from 0.01% to 50% by weight, in relation to the total weight of the composition.

10. The composition according to claim 1, wherein the weight ratio between polyol(s) and chitosan is between 0.1 and 5.

11. The composition according to claim 1, which comprises at least 30% by weight water with respect to the total weight of the composition and optionally at least one non-ionic surfactant.

12. The composition according to claim 1, which has a pH less than or equal to 7.

13. A method for making up and/or caring for the skin and/or the keratin appendages, wherein the composition according to claim 1 is applied to the skin and/or the keratin appendages.

14. The composition according to claim 1, wherein the alpha hydroxy acid is selected from lactic acid, citric acid and the salts and mixtures thereof.

15. The composition according to claim 1, wherein the alpha hydroxy acid (b) is present in a quantity ranging from 0.005 to 5% by weight with respect to the total weight of the composition.

16. The composition according to claim 1, wherein the dye (c) is selected from iron oxide, titanium dioxide and mixtures thereof.

17. The composition according to claim 1, wherein the polyol (d) is selected from glycerin, propylene glycol, dipropylene glycol, butylene glycol, hexylene glycol and mixtures thereof.

18. The composition according to claim 1, wherein the polyol (d) is present in a content ranging from 0.05% to 30% by weight, in relation to the total weight of the composition.

19. The composition according to claim 1, wherein the weight ratio between polyol(s) and chitosan is between 0.2 and 5.

20. The composition according to claim 1, which comprises at least 40% by weight water with respect to the total weight of the composition, and optionally at least one non-ionic surfactant selected from fatty acid and polyglycerol esters, sugar esters, poloxamers, polysorbates and mixtures thereof.
